(19) United States
(12) Patent Application Publication
(10) Pub. No.: US 2020/0013512 A1
(43) Pub. Date: Jan. 9, 2020

(54) SYSTEM AND METHOD FOR PROGRAMMATICALLY DETERMINING THE MORTALITY OF A NATURAL PERSON

(71) Applicants: Sahar M. Ismail, Arlington, TX (US); Michael T. Lester, Arlington, TX (US)

(72) Inventors: Sahar M. Ismail, Arlington, TX (US); Michael T. Lester, Arlington, TX (US)

(21) Appl. No.: 16/241,406

(22) Filed: Jan. 7, 2019

Related U.S. Application Data
(60) Provisional application No. 62/695,236, filed on Jul. 9, 2018.

(51) Int. Cl. G16H 50/30; G06F 17/18; G06F 16/903; G06F 17/27

(52) U.S. Cl. CPC G16H 50/30; G06F 17/2705; G06F 16/90344; G06F 17/18

(57) ABSTRACT

A method of determining a mortality score of a natural person, the method includes receiving electronic transmission of data from one or more sources into a data acquisition server; comparing, through an application server, the data to a database to determine if the data is new; and analyzing the data to determine the mortality score of the natural person through the application server and the user of an artificial intelligence engine associated with the application server; the mortality score represents a probability that the natural person is deceased.

### BACKGROUND

The present invention relates to a system and method for programmatically determining the mortality of a natural person.

Many activities and processes are triggered by the death of a natural person. These activities include, but are not limited to, payment of life insurance policies, freezing of bank accounts and credit cards, initiation of probate, termination of powers of attorney, etcetera. Today, notice or determination of the death of a natural person is still a manual, binary process. Determination of death by any entity is accomplished by the receipt of an official death certificate, notice via an obituary in a local newspaper, by the deceased person's social security number appearing in the U.S. Death Index maintained by the Social Security Administration, or some other similar method. All of these methods of mortality determination have the weakness that they are binary, i.e. the person is either dead or alive, and they are all decentralized and have a large time delay from the time of mortality until the time of notification. The U.S. Death Index itself has an almost six month delay in reporting. A bank, for example, might not know for months that someone is deceased because they didn't read a particular newspaper the day an obituary was posted and none of the deceased survivors knew that the deceased had an account at that bank, so no notification was made. This places companies who deal with natural persons as clients at risk for providing services or finances to people who are no longer living, and this in turn facilitates fraud and abuse.

The present invention comprises a method of determining the mortality of a natural person using input from a multitude of electronic sources and analyzing that input using an artificial intelligence engine to determine a mortality score which represents the likelihood of a natural person being deceased or being alive and existing as an animated being. Input sources include all current and future data sources which may contain data directly or indirectly related to the determination of a natural person's existence or demise.

### DESCRIPTION OF THE DRAWINGS

The novel features believed characteristic of the embodiments of the present application are set forth in the appended claims. However, the embodiments themselves, as well as a preferred mode of use, and further objectives and advantages thereof, will best be understood by reference to the following detailed description when read in conjunction with the accompanying drawings, wherein:

FIG. 1 is a flowchart of a data analysis associated with the system and method of the present application;

FIG. 2 is a flowchart of a data acquisition associated with the system and method of the present application; and

FIG. 3 is a simplified schematic of the system of the present application.

While the system and method of use of the present application is susceptible to various modifications and alternative forms, specific embodiments thereof have been shown by way of example in the drawings and are herein described in detail. It should be understood, however, that the description herein of specific embodiments is not intended to limit the invention to the particular embodiment disclosed, but on the contrary, the intention is to cover all modifications, equivalents, and alternatives falling within the spirit and scope of the present application as defined by the appended claims.

### DETAILED DESCRIPTION OF THE PREFERRED EMBODIMENT

Illustrative embodiments of the system and method of use of the present application are provided below. It will of course be appreciated that in the development of any actual embodiment, numerous implementation-specific decisions will be made to achieve the developer's specific goals, such as compliance with system-related and business-related constraints, which will vary from one implementation to another. Moreover, it will be appreciated that such a development effort might be complex and time-consuming, but would nevertheless be a routine undertaking for those of ordinary skill in the art having the benefit of this disclosure.

The terminology used herein is for the purpose of describing particular embodiments only and is not intended to be limiting of the invention. As used herein, the term “and/or” includes any and all combinations of one or more of the associated listed items. As used herein, the singular forms “a,” “an,” and “the” are intended to include the plural forms as well as the singular forms, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. Unless otherwise defined, all terms (including technical and scientific terms) used herein have the same meaning as commonly understood by one having ordinary skill in the art to which this invention belongs. It will be further understood that terms, such as those defined in commonly used dictionaries, should be interpreted as having a meaning that is consistent with their meaning in the context of the relevant art and the present disclosure and will not be interpreted in an idealized or overly formal sense unless expressly so defined herein.

The system and method of use will be understood, both as to its structure and operation, from the accompanying drawings, taken in conjunction with the accompanying description. Several embodiments of the system are presented herein. It should be understood that various components, parts, and features of the different embodiments may be combined together and/or interchanged with one another, all of which are within the scope of the present application, even though not all variations and particular embodiments are shown in the drawings. It should also be understood that the mixing and matching of features, elements, and/or functions between various embodiments is expressly contemplated herein so that one of ordinary skill in the art would appreciate from this disclosure that the features, elements, and/or functions of one embodiment may be incorporated into another embodiment as appropriate, unless described otherwise.

The preferred embodiment herein described is not intended to be exhaustive or to limit the invention to the precise form disclosed. It is chosen and described to explain the principles of the invention and its application and practical use to enable others skilled in the art to follow its teachings.

In describing the invention, it will be understood that a number of techniques and steps are disclosed. Each of these has individual benefit and each can also be used in conjunction with one or more, or in some cases all, of the other disclosed techniques. Accordingly, for the sake of clarity, this description will refrain from repeating every possible combination of the individual steps in an unnecessary fashion. Nevertheless, the specification and claims should be read with the understanding that such combinations are entirely within the scope of the invention and the claims.

The programmatic determination of the life or mortality of a natural person is discussed herein. In the following description, for purposes of explanation, numerous specific details are set forth in order to provide a thorough understanding of the present invention. It will be evident, however, to one skilled in the art that the present invention may be practiced without these specific details.

The present invention will now be described by referencing the appended figures representing preferred embodiments. This invention comprises the system, process, and methods necessary to calculate the probability that a natural person is alive or deceased. An anticipated and obvious embodiment of the invention is an online service that may be accessed directly or through an Application Programmer Interface (API).

The system will acquire information which pertains to a natural person through various electronic means and from various sources. This patent does not limit the sources those with a specific type, those with a specific data, or even to those in existence today. This patent describes a method for using data, however that data is available, and envisions using new sources of data as they become available. Likewise, the collection of data may be via “push” or “pull” technology where the system actively polls a data source for information or where the system accepts data sent to it, or broadcast, without initiation from the system.

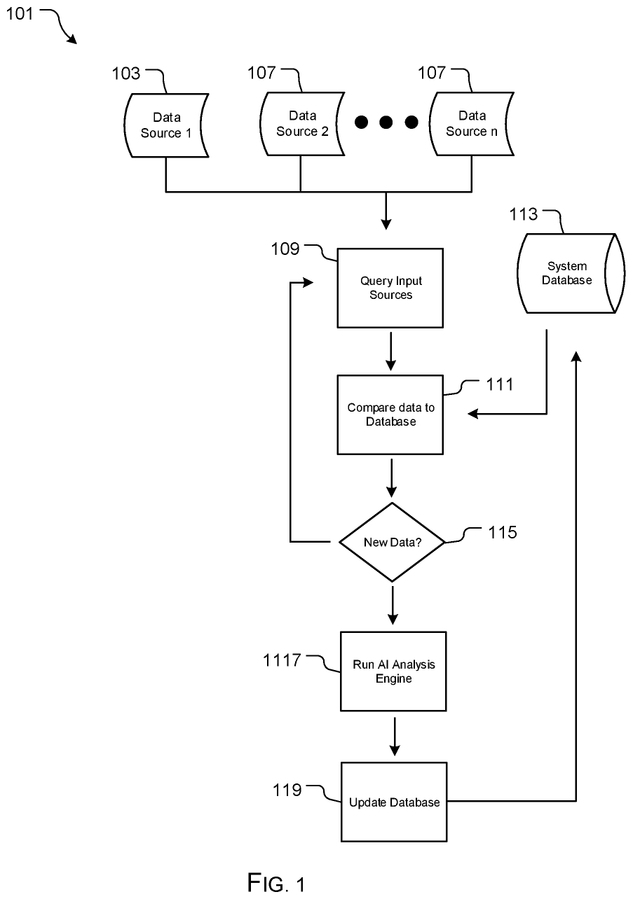

FIG. 1 depicts a flowchart 101 of data analysis of a system of the preferred embodiment at its highest level. The system includes a plurality of data sources 103, 105, 107 configured to be provided via one or more electronic devices as input sources. These data sources relate to any information that can be obtained on the death of a natural person. In the preferred embodiment, a query is generated either in an ad hoc, asynchronous fashion or as part of a scheduled batch processing. The system is configured to receive and query the input sources 109 while comparing the data sources 111 to a database 113 of information to determine a number of factors, such as the reliability of the data sources and whether or not the data has already been obtained. In the event of new data 115 the query process is repeated. For each subject (natural person or person of interest), identified by a plurality of means including name, birth date, sex, physical attributes, and various identification numbers, the Artificial Intelligence (AI) engine analyzes 117 all available information based on proprietary calculations and provides an output 119 which consists of a probability of whether or not that subject is currently living or deceased. The specifics of the AI code are confidential, proprietary, and outside of the scope of this patent application albeit to say that the AI engine will assign probabilistic aggregations to various events and datum in order to determine a meaningful and reproduceable output related to the subject's mortality.

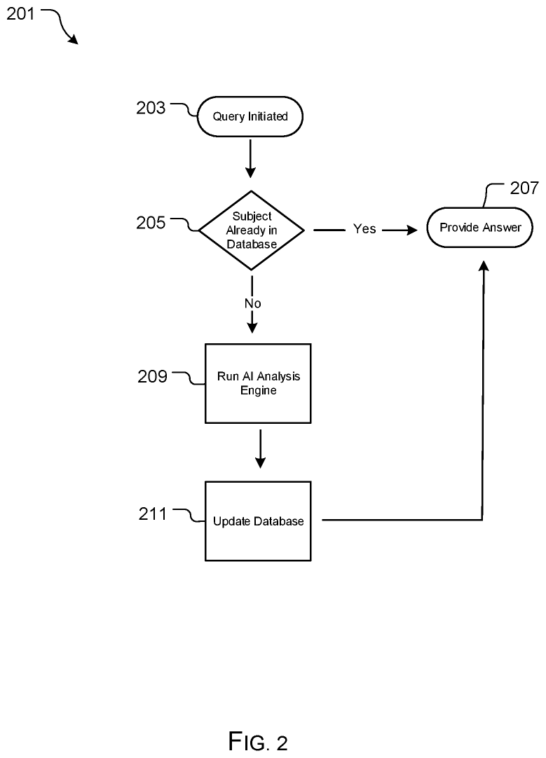

FIG. 2 is a flowchart 201 that shows the data acquisition portion of an embodiment of the system. This part of the embodiment will run continuously in a computer environment. The query will be initiated 203 and the system will access information from a wide variety of sources and categorize each datum with respect to that datum's relevance to a subject (natural person or person of interest). The system determines if the information is new and was not previously associated with a subject 205. If the information is not new, an output 207 is provided. If the information is new, the AI engine is invoked 209 to recalculate a mortality score for the subject. The new information and updated mortality score are then refreshed 211 in the database 207. This process continues in perpetuity to maintain accurate analysis and mortality scores for each subject.

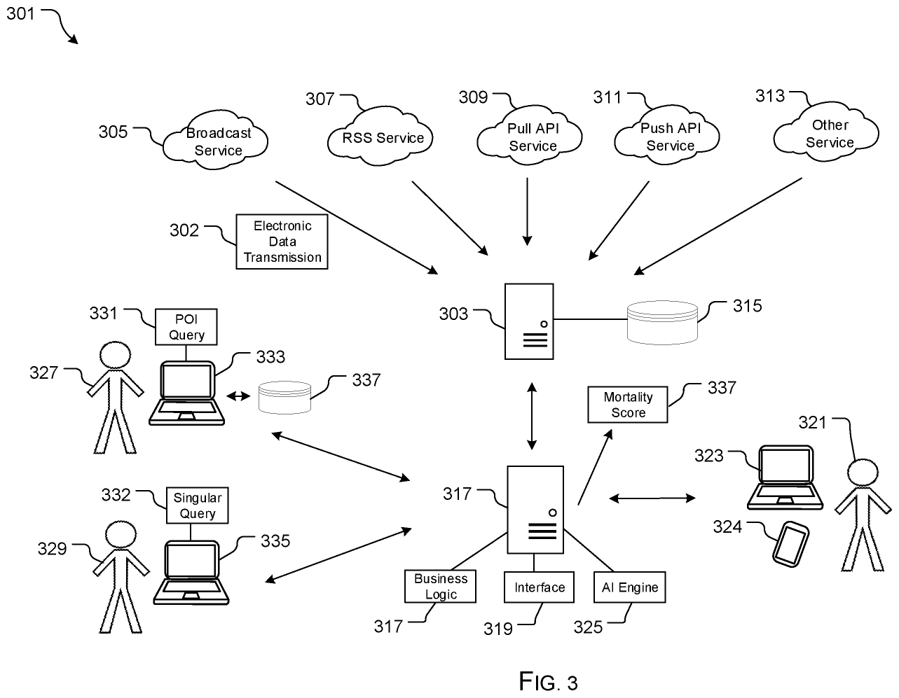

FIG. 3 shows a preferred embodiment of a method and system 301 of the present system. The system 301 comprises a data acquisition server 303 configured to receive electronic data transmission 302 from a plurality of services, including any of a broadcast service 305, an RSS service 307, a Pull API services 309, a Push API service 311, and any other service 313. The data is received by the data acquisition service, which is in communication and logically interconnected with a database 315.

In the embodiment, an application server 317 hosts a business logic 317 of the method and system, one or more interfaces 319 for receiving and sending data to an end user 321 or an end user's computer system 323, 324. The application server 317 further hosts the Artificial Intelligence Analysis Engine (AIAE) 325, and any other standard software application utilities such as, but not limited to, accounting, administrative control, reporting, etcetera.

In the preferred embodiment, one or more users 327, 329 can initiate queries 331, 332 to the application server either singularly in a synchronous, pseudo-real-time manner, or via an asynchronous upload of a list of Persons Of Interest (POI) through computing devices 333, 335. The application server will query the POI database 337 to determine if the POI is within the database 315. Based on the outcome of that internal query, the application server 317 and engine communicates with the Data Acquisition server 303/database 315 and engine to either initiate, or update, relevant information regarding the POI from query 331. That information is used as input to the artificial intelligence engine interface to calculate a mortality score 337 for the POI. The newly calculated score is stored in the POI database 337 and transmitted to the end user 321 either synchronously or asynchronously as previously configured by the end user.

It should be appreciated that the mortality score 337 provides relevant information to the end user to determine the likelihood that the person of interest is living or deceased.

In the preferred embodiment shown in FIG. 3, the portrayal of the application server, AIAE, Data acquisition Service, business logic, databases, and other operational systems needed to facilitate a stand-alone computing system as either separate or combined physical entities is for the ease of visualization. In an actual embodiment, the different logical units and databases may be co-located physically within a single unit.

Information obtained by the data acquisition server 303 may be acquired through a direct query of the external data source by the data acquisition engine, or by a passive analysis of data that is broadcast by the external data source or received by the data acquisition engine directly as part of a data transmission initiated by the external data source.

Information received by the data acquisition server 303 is parsed and assigned a credibility and relevance score by the AI engine. Credible and verifiable sources, such as the U.S. government, will receive a higher credibility score than sources that are less rigorous in their validation, such as Facebook or Wikipedia. Likewise, information that can be highly correlated to a subject, such as information tied to a unique identifier, like a social security number, will have a higher relevance than information that may apply to different subjects, such as a first and last name alone.

Potential data sources will include all sources of data which can be electronically accessed, or which may be reduced to electronic access, that contain information that the AI Analysis Engine might find useful in determining mortality. These sources include, but are not limited to online newspapers, obituary columns, government data bases, financial institutions, funeral homes, licensing authorities, social media, utility records, real estate records, stock records, etcetera. All data pertaining to a subject will be used to either substantiate life or substantiate mortality.

The AI engine of the system will analyze not only the data and its relevance and credibility scores, but also the temporal proximity of the information and its reception velocity. In other words, a datum received in close temporal proximity to other similar datum will score higher in credibility than the same data received temporally distant.

The system will return results to the calling interface, whether that interface be an API, web interface, database query, or other query method, in the form of a probabilistic score indicating the subjects likelihood of being alive or deceased. Amplifying data which supports the analysis may or may not be returned with the probabilistic score. In one embodiment of the system, the system may provide a authorized entity a notification if a subject's mortality probability exceeds a certain threshold.

The particular embodiments disclosed above are illustrative only, as the embodiments may be modified and practiced in different but equivalent manners apparent to those skilled in the art having the benefit of the teachings herein. It is therefore evident that the particular embodiments disclosed above may be altered or modified, and all such variations are considered within the scope and spirit of the application. Accordingly, the protection sought herein is as set forth in the description. Although the present embodiments are shown above, they are not limited to just these embodiments, but are amenable to various changes and modifications without departing from the spirit thereof.

1. A method of determining a mortality score of a natural person, the method comprising:
receiving electronic transmission of data from one or more sources into a data acquisition server;
comparing, through an application server, the data to a database to determine if the data is new; and
analyzing the data to determine the mortality score of the natural person through the application server and the user of an artificial intelligence engine associated with the application server;
wherein the mortality score represents a probability that the natural person is deceased.

2. The method of claim 1, further comprising:
assigning a credibility score and a relevance score to each datum by the artificial intelligence engine.

3. The method of claim 1, further comprising:
comparing a temporal proximity associated with each datum of the data.

4. The method of claim 1, wherein the receiving of electronic transmission of data is received through one or more services, including one or more of a broadcast service, an RSS service, a Pull API services, and a Push API service.

5. The method of claim 1, wherein analyzing the data comprises:
receiving data from a first source and a second source;
querying the first source and the second source;
comparing data received from the first source and the second source to the database;
running the artificial intelligence engine; and
updating the database based on results from the artificial intelligence engine.

6. The method of claim 1, further comprising:
receiving a query from a user into the application server from a computing device.

7. The method of claim 6, wherein the query includes an asynchronous upload of a list of persons of interest into the application server, the application server to compare a person of interest database to the database to determine if a person of interest as input by the user is within the database.

8. The method of claim 7, further comprising:
updating relevant information about the person of interest within the database.

9. The method of claim 6, wherein the query is provided singularly in a synchronous, pseudo-real-time manner.

10. The method of claim 1, wherein the data is received from one or more of an online newspaper, an obituary column, a government data base, a financial institution, a funeral home, a licensing authority, a social media, a utility records, a real estate records, and a stock records.
